(19) United States
(12) Patent Application Publication
(10) Pub. No.: US 2004/0132755 A1
(43) Pub. Date: Jul. 8, 2004

(54) Isoxazoles and uses thereof

(76) Inventors: Mark Ledeboer, Acton, MA (US); Brian Ledford, Attleboro, MA (US); Francesco G. Salituro, Marlboro, MA (US)

Correspondence Address: VERTEX PHARMACEUTICALS INC., 130 WAVERLY STREET, CAMBRIDGE, MA, 02139-4242, US

(21) Appl. No.: 10/657,811

(22) Filed: Sep. 8, 2003

Related U.S. Application Data
(60) Provisional application No. 60/408,813, filed on Sep. 6, 2002.

(51) Int. Cl. A61K 31/506; C07D 413/14

(52) U.S. Cl. 514/275; 544/331

(57) ABSTRACT

The present invention provides a compound of formula I:

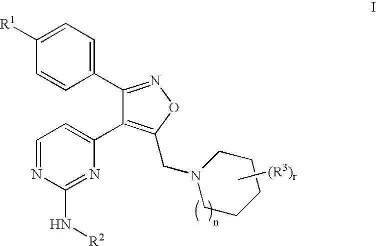

or a pharmaceutically acceptable salt thereof. These compounds are useful for the treatment of neurological, neurodegenerative, ischemic and inflammatory disorders. Accordingly, the invention also provides pharmaceutically acceptable compositions comprising the compounds of the invention and methods of utilizing those compounds and compositions in the treatment of neurological, neurodegenerative, ischemic and inflammatory disorders.

### CROSS-REFERENCE TO RELATED APPLICATIONS

The present application claims priority under 35 U.S.C. § 119(e) to U.S. Provisional Application No. 60/408,813, filed Sep. 6, 2002, entitled “Isoxazoles and Uses Thereof”, the entire contents of which are hereby incorporated by reference.

### TECHNICAL FIELD OF INVENTION

The present invention relates to isoxazoles useful for a variety of human diseases and conditions, such as ischemic disorders, reperfusion/ischemia in stroke, neurodegenerative disorders, neurological disorders, inflammatory diseases, heart disease, organ hypoxia, and thrombin-induced platelet aggregation to name a few. The invention also provides pharmaceutically acceptable compositions comprising the compounds of the invention and methods of using the compositions in the treatment of various disorders.

### BACKGROUND OF THE INVENTION

Stroke, which results from a reduction of, or disruption in, blood flow to the brain, is the third leading cause of death in the United States and other developed countries. Additionally, patients who survive a stroke typically have long-term disabilities including paralysis of the face or extremities, speech disorders, loss of bladder function, inability to swallow, or dementia.

Strokes are usually characterized as either ischemic (resulting from deficiency of oxygen in vital tissues) or hemorrhagic (resulting from a tear in the artery's wall that produces bleeding in the brain). The most common type of stroke, an ischemic stroke, causes over 80% of all strokes. It is believed that after oxygen deprivation occurs in an ischemic stroke, a cycle of events is triggered which ultimately leads to cell death. For example, it has been hypothesized that proteins, such as excitatory amino acids are released, which when overproduced, kill nerve cells. It is believed that these proteins open channels in the membranes that cover neurons allowing large amounts of calcium to flow in, and the calcium subsequently reacts within neurons to release harmful substances that damage cells. An additional hypothesis has suggested that PARP, an enzyme that ordinarily makes minor molecular repairs, responds to a substantial injury by taking up excessive ATP, thus resulting in additional cell death surrounding the original zone of cell death. Furthermore, it has been hypothesized that additional injury also results from downstream responses, such as an inflammatory reaction (see, del Zoppo et al. Brain Pathology, 2000, 10, 95-112).

Medical investigators have, for decades, unsuccessfully sought an effective early treatment for ischemic brain stroke. Clearly, the more quickly the ischemic brain tissue receives arterial blood the greater the chance that cell death and the resulting permanent injury can be reduced or prevented. Drugs that are currently used for the initial treatment of ischemic stroke include intravenous thrombolytics, such as t-PA (Activase®) or Streptokinase; and anti-clotting agents such as Ancrod, Asprin, Aggrenox, Thienopyridines, and Warfarin. Unfortunately, however, many of these agents are not effective in the treatment of ischemic stroke or are associated with severe side effects. For example, thienopyridines such as Ticlopidine (Ticlid®) have been associated with reversible lupus-like symptoms, reversible neutropenia and thrombocytopenia.

Considering the lack of currently available treatment options for stroke, it would be desirable to develop a safe and effective therapeutic agent capable of reducing or preventing ischemic injury, and/or capable of reducing or preventing injury resulting from downstream cellular responses (e.g., inflammatory response). More generally, it would also be desirable to develop a therapeutic agent useful for the treatment of other ischemic and inflammatory disorders.

### SUMMARY OF THE INVENTION

The present invention addresses this need by providing compounds of formula I:

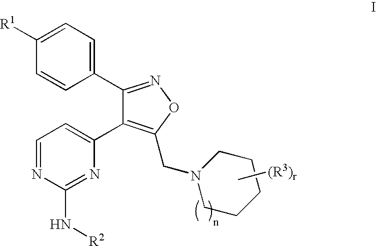

or a pharmaceutically acceptable salt thereof, wherein R¹, R², R³, n and r are as defined below.

The compounds of this invention, and pharmaceutically acceptable compositions thereof, are useful for treating, preventing, or lessening the severity of a variety of disorders, including neurodegenerative disorders, neurological disorders, inflammatory disorders, ischemic disorders, reperfusion/ischemia in stroke, heart disease, allergic disorders, organ hypoxia, and thrombin-induced platelet aggregation to name a few.

### DETAILED DESCRIPTION OF THE INVENTION

I. Description of Compounds of the Invention:

The present invention relates to a compound of formula I:

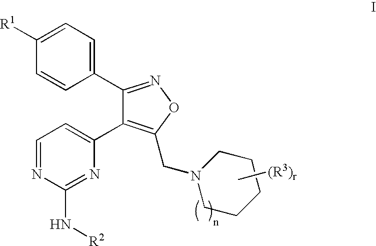

or a pharmaceutically acceptable salt thereof, wherein:

R¹ is hydrogen or halogen;

R² is substituted or unsubstituted cycloalkyl;

each occurrence of R³ is independently halogen, alkyl, —(CH₂)ₘOR⁴, —(CH₂)ₘSR⁴, —(CH₂)ₘN(R⁴)₂, —(CH₂)ₘNR⁴C(O)R⁴, —(CH₂)ₘNR⁴C(O)N(R⁴)₂, —(CH₂)ₘNR⁴CO₂R⁴, —(CH₂)ₘCO₂R⁴, —(CH₂)ₘC(O)R⁴, —(CH₂)ₘC(O)N(R⁴)₂, —(CH₂)ₘOC(O)N(R⁴)₂, —(CH₂)ₘS(O)₂R⁴, —(CH₂)ₘSO₂N(R⁴)₂, —(CH₂)ₘS(O)R⁴, —(CH₂)ₘNR⁴SO₂N(R⁴)₂, —(CH₂)ₘNR⁴SO₂R⁴, —(CH₂)ₘC(═S)N(R⁴)₂, wherein m is 0, 1 or 2 and R⁴ is hydrogen or alkyl;

r is 0, 1 or 2; and

n is 0, 1 or 2.

As used herein, the following definitions shall apply unless otherwise indicated.

The phrase “optionally substituted” is used interchangeably with the phrase “substituted or unsubstituted.” Unless otherwise indicated, an optionally substituted group may have a substituent at each substitutable position of the group, and each substitution is independent of the other.

The terms “alkyl”, “alkoxy”, “hydroxyalkyl”, “alkoxyalkyl”, and “alkoxycarbonyl”, used alone or as part of a larger moiety include both cyclic and acyclic, substituted and unsubstituted, and straight and branched chains containing one to seven carbon atoms, or preferably one to four carbon atoms, and at least two carbon atoms and one double bond in the case of alkenyl and at least two carbon atoms and one triple bond in the case of alkynyl. In certain embodiments, a cycloalkyl group preferably contains five, six or seven carbon atoms and may be monocyclic or bicyclic. Furthermore, a cycloalkyl group may contain one or more substituents. Suitable substituents (R⁵) for replacement of one or more hydrogen atoms on the saturated carbon of a cycloalkyl ring (as defined by R²) include one or more independent occurrences of: halogen, alkyl, —(CH₂)_{q}OR⁶, —(CH₂)_{q}SR⁶, —(CH₂)_{q}N(R⁶)₂, —(CH₂)_{q}NR⁶C(O)R⁶, —(CH₂)_{q}NR⁶C(O)N(R⁶)₂, —(CH₂)_{q}NR⁶CO₂R⁶, —(CH₂)_{q}CO₂R⁶, —(CH₂)_{q}C(O)R⁶, —(CH₂)_{q}C(O)N(R⁶)₂, —(CH₂)_{q}OC(O)N(R⁶)₂, —(CH₂)_{q}S(O)₂R⁶, —(CH₂)_{q}SO₂N(R⁶)₂, —(CH₂)_{q}S(O)R⁶, —(CH₂)_{q}NR⁶SO₂N(R⁶)₂, —(CH₂)_{q}NR⁶SO₂R⁶, —(CH₂)_{q}C(═S)N(R⁶)₂, wherein q is 0, 1 or 2 and each occurrence of R⁶ is independently hydrogen or alkyl.

A combination of substituents or variables is permissible only if such a combination results in a stable or chemically feasible compound. A stable compound or chemically feasible compound is one that is not substantially altered when kept at a temperature of 40° C. or less, in the absence of moisture or other chemically reactive conditions, for at least a week.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention. Unless otherwise stated, structures depicted herein are also meant to include compounds that differ only in the presence of one or more isotopically enriched atoms. For example, compounds having the present structures except for the replacement of a hydrogen by a deuterium or tritium, or the replacement of a carbon by a ¹³C- or ¹⁴C-enriched carbon are within the scope of this invention. Such compounds are useful, for example, as analytical tools or probes in biological assays.

II. Description of Certain Exemplary Compounds:

In certain exemplary embodiments, for compounds of formula I described directly above, R¹ is hydrogen or fluorine; R² is substituted or unsubstituted cycloalkyl; r is 0 or 1; R³ is alkyl, or —(CH₂)ₘOR⁴; m is 0, 1 or 2; R⁴ is hydrogen or alkyl; and n is 0, 1 or 2.

In certain other exemplary embodiments, R² is substituted or unsubstituted cyclohexyl or norbornyl and thus compounds having the structures II and III are provided:

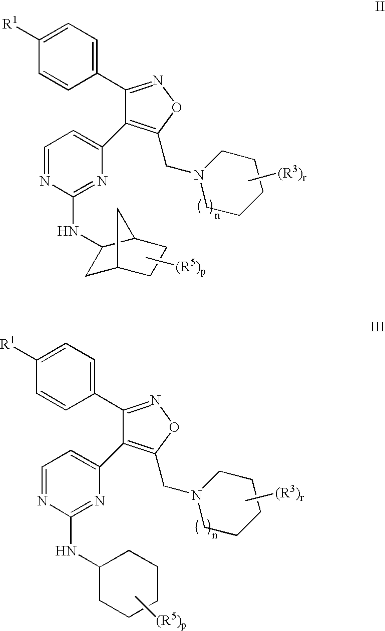

wherein R¹, R³, r and n are as defined above and in subsets herein; each occurrence of R⁵ is independently halogen, alkyl, —(CH₂)_{q}OR⁶, —(CH₂)_{q}SR⁶, —(CH₂)_{q}N(R⁶)₂, —(CH₂)_{q}NR⁶C(O)R⁶, —(CH₂)_{q}NR⁶C(O)N(R⁶)₂, —(CH₂)_{q}NR⁶CO₂R⁶, —(CH₂)_{q}CO₂R⁶, —(CH₂)_{q}C(O)R⁶, —(CH₂)_{q}C(O)N(R⁶)₂, —(CH₂)_{q}OC(O)N(R⁶)₂, —(CH₂)_{q}S(O)₂R⁶, —(CH₂)_{q}SO₂N(R⁶)₂, —(CH₂)_{q}S(O)R⁶, —(CH₂)_{q}NR⁶SO₂N(R⁶)₂, —(CH₂)_{q}NR⁶SO₂R⁶, —(CH₂)_{q}C(═S)N(R⁶)₂, wherein q is 0, 1 or 2 and each occurrence of R⁶ is independently hydrogen or alkyl; and p is 0, 1 or 2.

Certain subclasses of the foregoing compounds are described in more detail below. It will be appreciated that, for each of the compounds generally described above (formula I) and classes thereof, (e.g., formulas II and III), any combination of the subsets set forth below may be utilized to describe exemplary subclasses of the invention. In particular, certain preferred subclasses include, but are not limited to the following:

i) compounds as described generally above and in classes and subclasses herein where R¹ is F;

ii) compounds as described generally above and in classes and subclasses herein where R¹ is H;

iii) compounds as described generally above and in classes and subclasses herein where R² is substituted or unsubstituted cyclohexyl;

iv) compounds as described generally above and in classes and subclasses herein where R² is substituted or unsubstituted norbornyl;

v) compounds as described generally above and in classes and subclasses herein where R³ is alkyl, OH, CH₂OH, or alkoxy;

vi) compounds as described generally above and in classes and subclasses herein where n is 0;

vii) compounds as described generally above and in classes and subclasses herein where n is 1;

viii) compounds as described generally above and in classes and subclasses herein where n is 2;

ix) compounds of formula II where p is 0;

x) compounds of formula III where p is 0;

xi) compounds of formula III where p is 1;

xii) compounds of formula III where p is 2;

xiii) compounds of formula II or III where p is 0 or 1 and R⁵ is independently OH, or alkyl; and

xiv) compounds as described generally above and in classes and subclasses herein where r is 0 or 1.

In certain preferred embodiments, compounds have the formula II and R¹ is F or H; p is 0; n is 0 or 1; r is 0 or 1; and R³ is OH, CH₂OH, alkyl or alkoxy.

In certain other preferred embodiments, compounds have the formula III and R¹ is F or H; p is 0, 1 or 2; each occurrence of R⁵ is independently alkyl, OH, CH₂OH or alkoxy; n is 0 or 1; r is 0 or 1; and R³ is OH, CH₂OH, alkyl or alkoxy.

Representative examples of compounds of formula I are set forth below in Table 1.

TABLE 1
Examples of Compounds of Formula I:

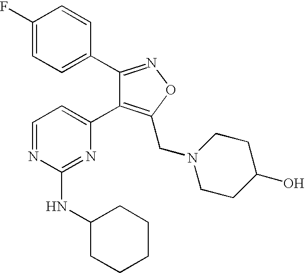

I-1

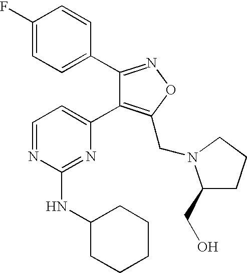

I-2

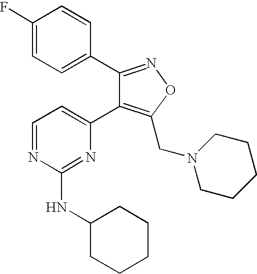

I-3

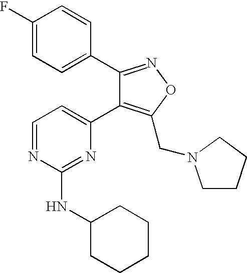

I-4

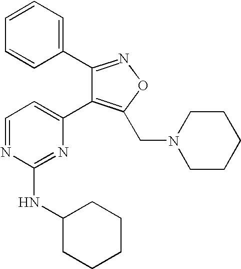

I-5

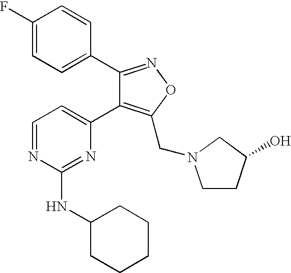

I-6

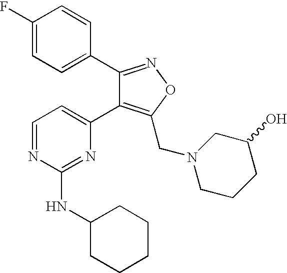

I-7

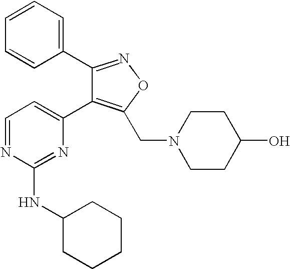

I-8

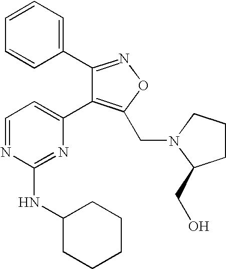

I-9

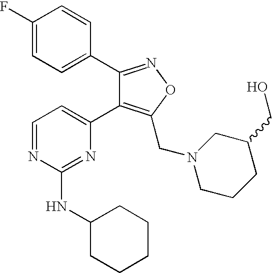

I-10

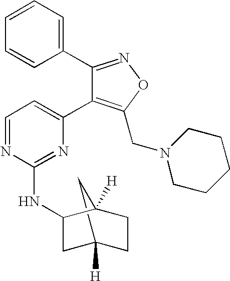

I-11

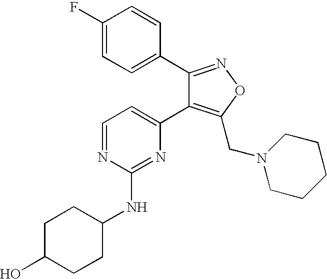

I-12

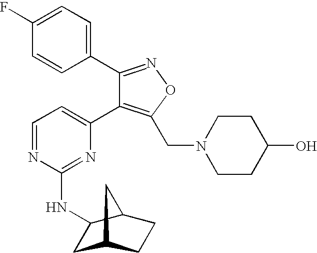

I-13

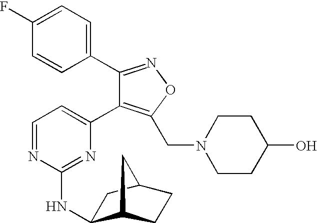

I-14

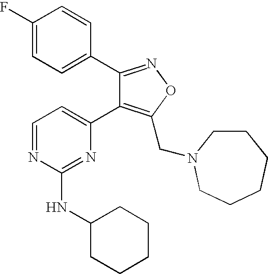

I-15

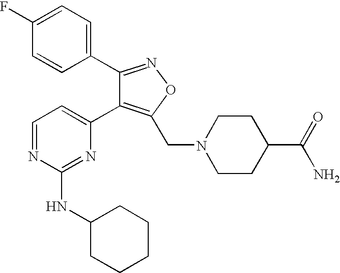

I-16

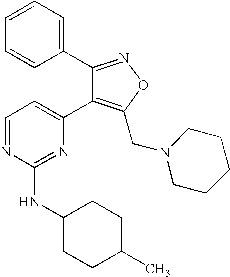

I-17

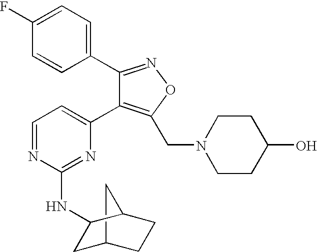

I-18

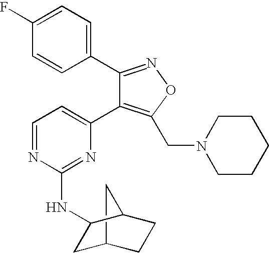

I-19

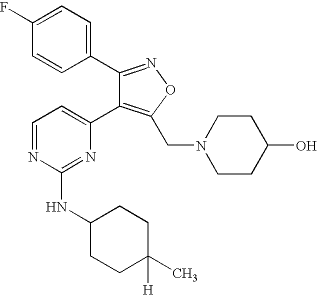

I-20

III. General Synthetic Methodology:

The compounds of this invention may be prepared in general by methods known to those skilled in the art for analogous compounds, as illustrated by the general scheme below, and the preparative examples that follow.

Scheme 1 below shows a general method for preparing compounds of formula I. In general, as depicted below, compounds depicted below (e.g., 6A, 7A, 10A and 11A) are useful intermediates in forming a variety of compounds of formulas I, II and III using methods known to one of skill in the art. Additionally, methods for the synthesis of these compounds are described generally in PCT publication WO 01/12621, the entire contents of which are hereby incorporated by reference. Scheme 2 depicts a general method for the synthesis of certain exemplary compounds of formulas II and III (directed to norbornyl and cyclohexyl derivatives) where the fluoro substituted chloroxime intermediate, and unsubstituted and hydroxy-substituted piperidinyl intermediates are utilized.

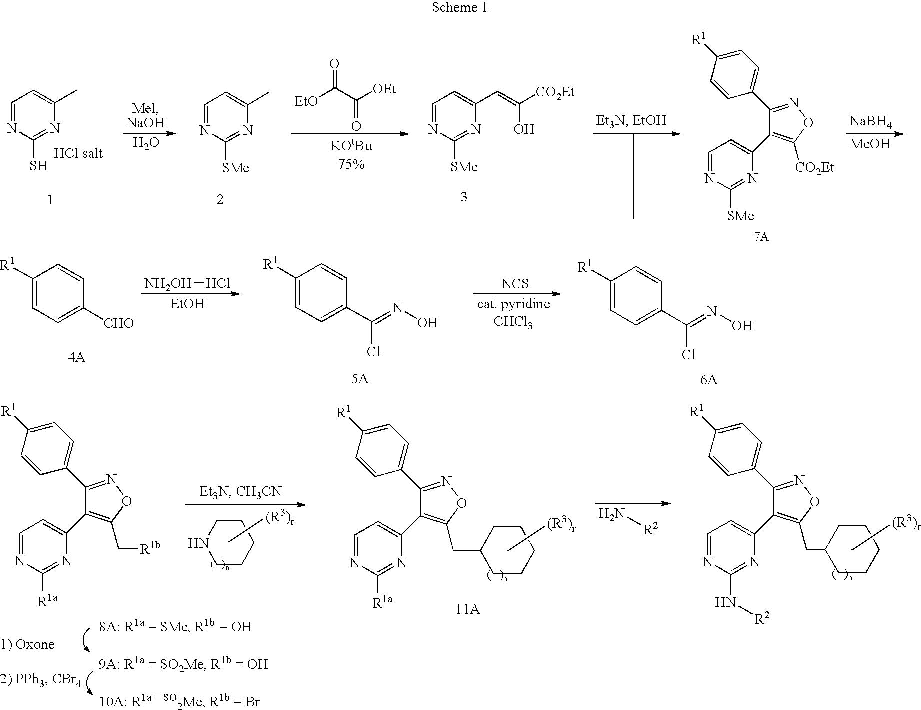

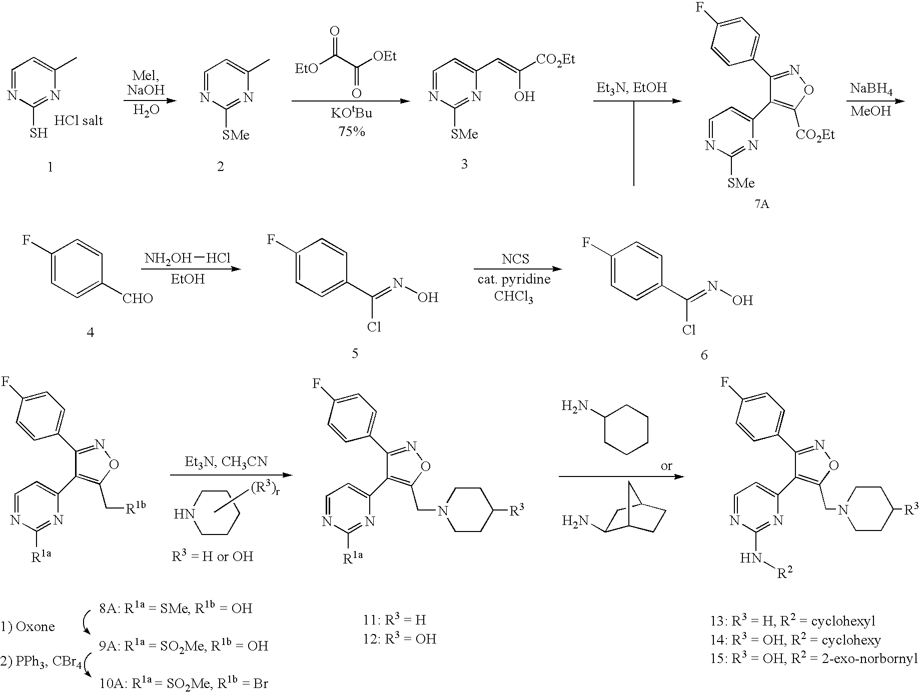

IV. Uses of Compounds of the Invention:

The compounds of the invention unexpectedly and surprisingly exhibit increased potency in the protection of neuronal cells against ischemic injury and as inhibitors in in vitro CNS inflammation assays. The activity of compounds utilized in this invention may be assayed in vitro, in vivo or in a cell line according to methods known in the art. Exemplary in vitro assays include in vitro ischemia (OGD) assays, and in vitro CNS inflammation assays. In vivo assays include rat MCAO (middle cerebral artery occlusion) efficacy studies, as described in more detail herein.

According to another embodiment, the invention provides a composition comprising a compound of this invention or a pharmaceutically acceptable salt thereof and a pharmaceutically acceptable carrier, adjuvant, or vehicle. The amount of compound in the compositions of this invention is such that is effective to treat, prevent, or reduce the severity of an ischemic, inflammatory, neurodegenerative or neurological disorder in a patient. Preferably the composition of this invention is formulated for administration to a patient in need of such composition. Most preferably, the composition of this invention is formulated for oral administration to a patient.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N⁺(C₁₋₄ alkyl)₄ salts. This invention also envisions the quaternization of any basic nitrogen-containing groups of the compounds disclosed herein. Water or oil-soluble or dispersible products may be obtained by such quaternization.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, the pharmaceutically acceptable compositions of this invention are formulated for oral administration.

The amount of the compounds of the present invention that may be combined with the carrier materials to produce a composition in a single dosage form will vary depending upon the host treated, the particular mode of administration. Preferably, the compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Depending upon the particular condition, or disease, to be treated or prevented, additional therapeutic agents, which are normally administered to treat or prevent that condition (or an associated side effect or disorder), may also be present in the compositions of this invention. As used herein, additional therapeutic agents that are normally administered to treat or prevent a particular disease, or condition, are known as “appropriate for the disease, or condition, being treated”.

For example, known treatments for stroke include Activase®, a recombinant, or genetically engineered, tissue plasminogen activator (rt-PA), heparin, glutamate antagonists, calcium antagonists, opiate antagonists, GABA agonists and antioxidants.

Other examples of agents the compounds of this invention may also be combined with include, without limitation: treatments for Alzheimer's Disease such as Aricept® and Excelon®; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

According to another embodiment, the invention relates to a method of treating, preventing or lessening the severity of a neurological, neurodegenerative, ischemic or inflammatory disorder comprising the step of administering a compound of this invention, or a composition comprising said compound to a subject, preferably a mammal and more preferably a human. In certain preferred embodiments, the invention relates to methods for treating, preventing or lessening the severity of ischemic disorders and most preferably relates to methods for treating, preventing or lessening the severity of stroke.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

The term “ischemic disorder”, as used herein, includes any disease or condition affecting the blood vessels of an organ. In certain preferred embodiments, an ischemic disorder includes any disease or condition affecting the blood vessels of the brain (e.g., stroke and transient ischemic attacks, to name a few).

Neurodegenerative diseases which may be treated or prevented by the compounds of this invention include, but are not limited to, Alzheimer's disease, Parkinson's disease, cerebral ischemias or neurodegenerative disease caused by traumatic injury.

Exemplary schemic disorders and diseases which may be treated or prevented by the compounds of this invention include, but are not limited to, stroke and transient ischemic attacks.

Inflammatory diseases which may be treated or prevented by the compounds of this invention include, but are not limited to, acute pancreatitis, chronic pancreatitis, asthma, allergies, and adult respiratory distress syndrome.

Autoimmune diseases which may be treated or prevented by the compounds of this invention include, but are not limited to, glomerulonephritis, rheumatoid arthritis, systemic lupus erythematosus, scleroderma, chronic thyroiditis, Graves' disease, autoimmune gastritis, diabetes, autoimmune hemolytic anemia, autoimmune neutropenia, thrombocytopenia, atopic dermatitis, chronic active hepatitis, myasthenia gravis, multiple sclerosis, inflammatory bowel disease, ulcerative colitis, Crohn's disease, psoriasis, or graft vs. host disease.

In an alternate embodiment, the methods of this invention that utilize compositions that do not contain an additional therapeutic agent, comprise the additional step of separately administering to said patient an additional therapeutic agent. When these additional therapeutic agents are administered separately they may be administered to the patient prior to, sequentially with or following administration of the compositions of this invention.

The compounds of this invention or pharmaceutically acceptable compositions thereof may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a compound as described herein. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition. Implantable devices coated with a compound of this invention are another embodiment of the present invention.

In order that the invention described herein may be more fully understood, the following examples are set forth. It should be understood that these examples are for illustrative purposes only and are not to be construed as limiting this invention in any manner.

### EXAMPLES

As used herein,the term “Rₜ(min)” refers to the HPLC retention time, in minutes, associated with the compound using the HPLC method specified. Unless otherwise indicated, the BPLC methods utilized to obtain the reported retention times are as follows:

Method-A: Column: Lightning, 2.1×50 mm; Gradient: 100% water (0.1% TFA)→100% CH₃CN (0.1% TFA) over 4 minutes; Flow rate: 0.8 mL/minute.

Method-B: Column: Lightning, 2.1×50 mm; Gradient: 90% water (0.1% TFA)→90% CH₃CN (0.1% TFA) over 4 minutes; Flow rate: 0.8 mL/minute.

Method-C: Column: YMC ODS-AQ, 30×150 mm; Gradient: 10%→90% CH₃CN/water (0.01% TFA) over 8 minutes; Flow rate: 1 mL/minute; Detection: 210, 220, 254, 280, or 300 nm.

Method-D: Column: Diacell Chiralpak OJ-H 250×4.6 mm; Isocratic: 5% MeOH/CO₂; Pressure: 200 bar; Temp: 40° C.; Flow rate: 2 mL/minute; Detection: 220 nm.

### Example 1

### Preparation of Fluoro Substituted Chlorooxime 6

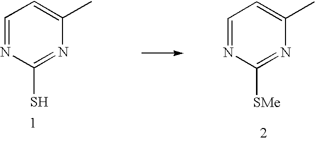

A) Preparation of 2: In a 5 L three-necked round-bottomed flask equipped with an overhead stirrer and reflux condenser, 497.6 g (3.06 mol) of the hydrochloride salt of the thiol pyrimidine 1 was suspended in 1 L of MeOH. A 6N NaOH solution (1.2 L, excess) was added and the mixture was stirred for 15 minutes. The solution was heated to reflux and methyl iodide (192 mL, 3.08 mol) was added in portions. The mixture was refluxed for two hours, cooled, and extracted with CH₂Cl₂. The extract was dried (MgSO₄) and evaporated in vacuo to afford 347.3 g (81%) of the sulfide as a light yellow oil.

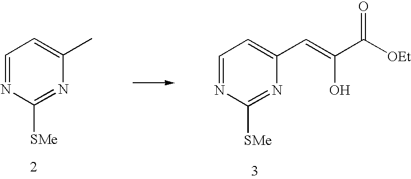

B) Preparation of 3: In a 5 L three-necked round-bottomed flask equipped with an overhead stirrer, 4-methyl-2-thiomethylpyrimdine (344.5 g, 2.46 mol) was dissolved in 2 L of THF. Diethyl oxalate (334 mL, 2.46 mol) was added. Potassium t-butoxide (286.22 g, 2.55 mol) was added in 50 g portions. Each portion produced a mild exotherm. The mixture rapidly turned brown and a yellow precipitate eventually formed forming a thick mixture. The reaction was poured into 10% aqueous NH₄Cl and extracted with CH₂Cl₂. The extract was dried (MgSO₄) and filtered through a plug of silica gel. The plug was eluted with 10% EtOAc/CH₂Cl₂. The filtrate was evaporated to afford a brown solid. The solid was dissolved in a minimal amount of CH₂Cl₂. Hexane was added and the solution was cooled to precipitate a solid that was filtered. The filtrate was evaporated and the precipitation process was repeated to afford a total of 401.5 g (60%) of the ketoester 3.

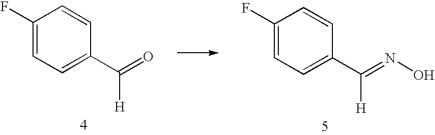

C) Preparation of 5: In a 3 L three-necked round-bottomed flask equipped with an overhead stirrer, 566.6 g (4.07 mol) of 4-fluorobenzaldehyde was dissolved in 2 L of MeOH. To this solution was added 565.0 g (6.89 mol) of sodium acetate. The solid did not completely dissolve. Hydroxylamine hydrochloride (304.42 g, 4.38 mol) was added in 50 g portions. Each addition of hydroxylamine hydrochloride resulted in a mild exotherm. A white solid precipitated during the reaction although the mixture never became very thick. The mixture was stirred for an hour. Water was added which initially solubilized the precipitate. The addition of more water precipitated a white crystalline solid that was filtered, washed with water, and dried in vacuo to afford 558.3 g (99%) of the oxime 5.

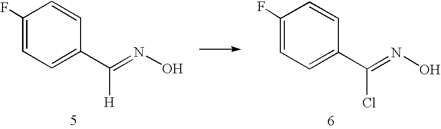

D) Preparation of fluoro substituted chlorooxime 6:

In a 3 L three-necked round-bottomed flask equipped with an overhead stirrer, 558.3 g (4.01 mol) of the oxime 5 was dissolved in 2 L of DMF. N-Chlorosuccinimide (581.6 g, 4.36 mol) was added in 30 g portions. An induction period was observed before a reaction took place. A mild exotherm was observed when the reaction started and with each addition of NCS. The reaction was stirred overnight and poured into water precipitating a white solid. The solid was filtered, washed with water, and evaporated in vacuo to afford 309.2 g (44%) of the chlorooxime 6 as a white solid.

### Example 2

### Preparation of Bromide (10)

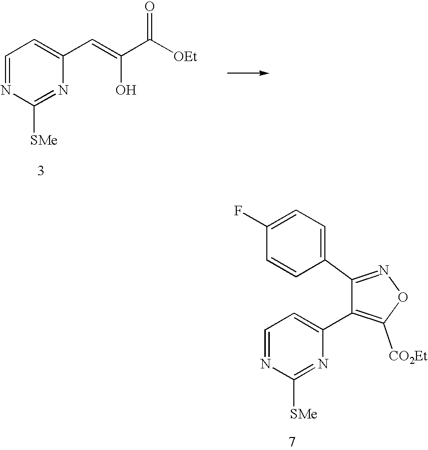

A) Preparation of 7: In a 1 L round-bottomed flask, 24.6 g (90.6 mMol) of the ketoester 3 was dissolved in 300 mL of ethanol. To the solution was added 18.6 g (107 mMol) of chlorooxime 6. Triethylamine (53 mL, 380 mMol) was added dropwise to the solution forming a yellow color and producing a mild exotherm. The solution was stirred until the ketoester was no longer evident by TLC (˜one hour). The mixture was poured into water and extracted with CH₂Cl₂. The extract was dried and filtered over a plug of silica gel. The plug was eluted with 25% EtOAc/CH₂Cl₂. The filtrate was evaporated in vacuo to afford the crude product as a yellow solid. The solid was dissolved in a minimal amount of CH₂Cl₂. Hexane was added and the solution was cooled precipitating 29.5 g (90%) of the isoxazole 7 as a yellow solid.

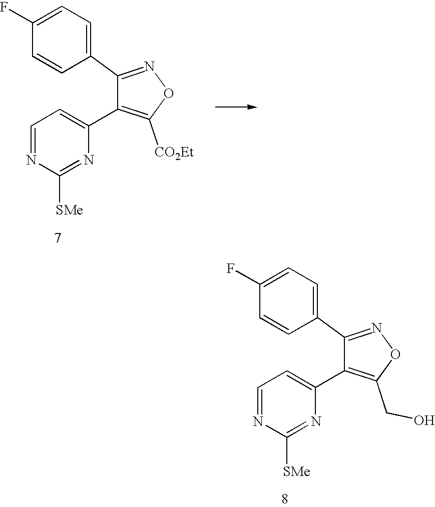

B) Preparation of 8: In a 5 L three-necked round-bottomed flask, the ester 7 (278.7 g, 773 mMol) was dissolved in 2 L of THF. Methanol (1 L) was added. Sodium borohydride (63.70 g, 1.67 mol) was added in 10 g portions. Each addition of the borohydride produced some foaming and copious amounts of gas evolution. The solution was stirred for 2 hours. TLC showed a mixture of the product 8 and a lower Rf spot. The solution was poured carefully into 1N HCl and extracted with CH₂Cl₂. The extract was dried (MgSO₄) and evaporated in vacuo. The oil was redissolved in a minimal amount of CH₂Cl₂, hexane was added and the solution was cooled to precipitate 58 g of the alcohol 8 as a white solid. The filtrate was concentrated in vacuo and filtered over a plug of silica gel. Elution of the plug with 5% EtOAc/CH₂Cl₂ selectively eluted the alcohol 8 away from the by-product. Another 48 g of the alcohol was recovered from the plug to produce 106 g (42%) total of the product.

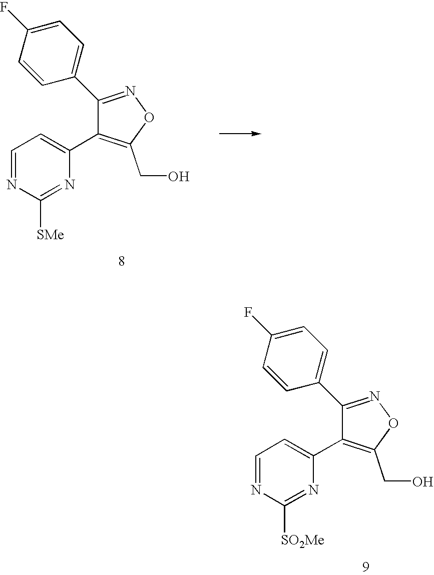

C) Preparation of 9: To a stirred solution of 8 (31.74 g, 100 mMol) in methanol (1.5 L) at ambient temperature was added a solution of Oxone® (135 g, 220 mMol) in water (750 mL) via drop-funnel over 45 min. The resulting reaction mixture was stirred overnight. Water was added and the mixture was extracted with CH₂Cl₂ (2×1 L). The combined organic phase was dried (MgSO₄), filtered and concentrated to provide the 9 (31.96 g, 91.48 mMol, 91%) as a white solid.

¹H NMR (CDCl₃, 500 MHz) δ 8.72 (d, 1H, J=5.4 Hz), 7.52-7.48 (two d, 2H, J=8.6 and 8.6 Hz), 7.28-7.22 (two d, 2H, J=8.4 Hz), 7.16 (d, 1H, J=5.4 Hz), 4.98 (s, 2H), 3.37 (s, 3H).

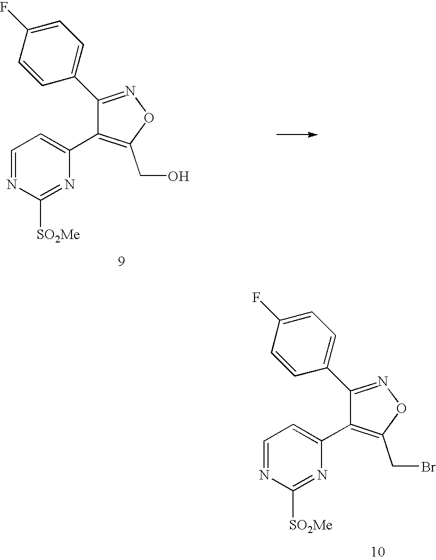

D) Preparation of 10: To a stirred solution of 9 (31.96 g, 91.48 mMol) in CH₂Cl₂ (500 mL) at ambient temperature was added CBr₄ (42.4 g, 128 mMol) followed by PPh₃ (28.8 g, 110 mMol). A slight exotherm was observed. After 20 min., additional PPh₃ (2.00 g) was added. After 2 h., the reaction was concentrated. The resulting material was picked up in EtOH (500 mL) and stirred. The desired product (10) precipitated from solution and was collected and rinsed with additional EtOH to provide 32.3 g (78.7 mMol, 86%) of an off-white solid.

¹H NMR (CDCl₃, 500 MHz) δ 8.78 (d, 1H, J=5.3 Hz), 7.52-7.48 (two d, 2H, J=8.8 and 8.8 Hz), 7.25-7.14 (two d, 2H, J=8.5 Hz), 7.17 (d, 1H, J=5.3 Hz), 4.98 (s, 2H), 3.39 (s, 3H).

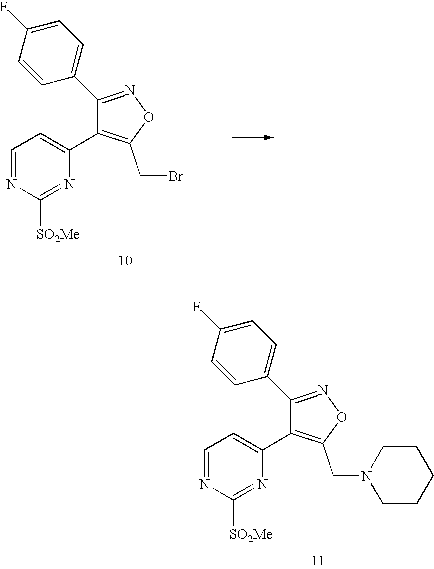

### Example 3

### Preparation of Piperidinyl Intermediate 11

To a stirred solution of 10 (1.66 g, 4.03 mMol) in CH₃CN (20 mL) at ambient temperature was added piperidine (0.406 mL, 4.11 mMol) followed by Et₃N (0.573 mL, 4.11 mMol). After 60 min, the reaction was concentrated, partitioned between saturated NaHCO₃ (50 mL) and CH₂Cl₂ (30 mL). The aqueous layer was extracted with additional CH₂Cl₂ (2×20 mL). The combined organic layer was dried (MgSO₄), filtered through SiO₂ and concentrated to provide 1.50 g (3.60 mMol, 89%) of a colorless oil.

¹H NMR (CDCl₃, 500 MHz) δ 8.82 (d, 1H, J=5.2 Hz), 7.62 (d, 1H, J=5.2 Hz), 7.51-7.47 (two d, 2H, J=8.8 and 8.8 Hz), 7.18-7.14 (two d, 2H, J=8.6 and 8.6 Hz), 3.94 (s, 2H), 3.14 (s, 3H), 2.54 (br m, 4H), 1.57 (m, 4H), 1.44 (m, 2H).

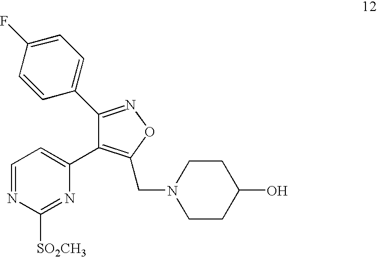

### Example 4

### Preparation of Hydroxy-Substituted Piperidinyl Intermediate 12

To a stirred solution of 10 (22.7 g, 55.2 mMol) in CH₃CN (220 mL) at ambient temperature was added 4-hydroxypiperidine (5.58 g, 55.2 mMol) followed by Et₃N (8.08 mL, 58.0 mMol). After 90 min, the reaction was concentrated, partitioned between saturated NaHCO₃ and CH₂Cl₂. The aqueous layer was extracted with additional twice more with CH₂Cl₂. The combined organic layer was dried (Na₂SO₄), filtered through SiO₂ and concentrated to provide 23.7 g (54.7 mMol, 99.2% yield) of a colorless foam.

¹H NMR (CDCl₃, 500 MHz) δ 8.79 (d, 1H, J=5.2 Hz), 7.51-7.47 (two d, 2H, J=8.6 Hz and 8.6 Hz), 7.46 (d, 1H, J=5.3 Hz), 7.20-7.15 (two d, 2H, J=8.6 and 8.6 Hz), 4.04 (s, 2H), 3.74 (m, 1H), 3.21 (s, 3H), 2.89 (m, 2H), 2.40 (m, 2H), 1.89 (m, 2H), 1.39 (d, 1H, J=4.7 Hz); HPLC (Method A) tᵣ=2.65 min; MS (ES⁺): m/z 433.1 (M+H).

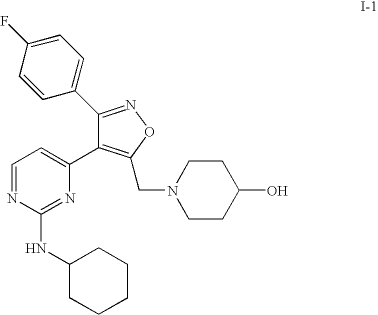

### Example 5

### Preparation of I-1

A stirred solution of 12 (24.8 g, 57.3 mMol) and cyclohexylamine (13.1 mL, 115 mMol) in DMSO (100 mL) was heated to 75° C. for 4.5 h. The reaction was allowed to cool to ambient temperature. An additional equivalent of cyclohexylamine (6.5 mL, 57 mMol) was added and the solution was stirred for 16 h during which time a white precipitate formed. The solid was collected by filtration and rinsed with several portions of EtOAc. The mother liquor was diluted with CH₂Cl₂, washed with water, saturated NaHCO₃, and brine. The organic phase was dried (MgSO₄), filtered and concentrated. The resulting solid was suspended in a small portion of hot EtOAc and filtered. The combined solid was stirred in EtOAc (100 mL) for 16 h and filtered again. The filtrate was rinsed with additional EtOAc. The resulting solid was dried in vacuo to provide 24.6 g (54.5 mMol, 95%) of a white amorphous powder.

¹H NMR (CDCl₃, 500 MHz) δ 8.18 (d, 1H, J=4.9 Hz), 7.51-7.48 (two d, 2H, J=8.8 Hz and 8.8 Hz), 7.11-7.07 (two d, 2H, J=8.7 and 8.7 Hz), 6.38 (d, 1H, J=4.0 Hz), 5.02 (d, 1H, J=7.9 Hz), 3.99 (s, 2H), 3.74-3.62 (br m, 2H), 2.84 (m, 2H), 2.34 (m, 2H), 1.93-1.88 (m, 4H), 1.72 (m, 2H), 1.61 (m, 4H), 1.42 (br s, 1H), 1.38-1.12 (m, 6H).

Bis HCl salt: A solution of I-1 (2.88 g, 6.38 mMol) in MeOH—CH₂Cl₂ (1:2) was treated with HCl (6.4 mL, 4 M in dioxane). After 2 minutes, Et₂O (50 mL) was added and a precipitate slowly formed. After stirring an additional 20 minutes, Et₂O (100 mL) was added and the solution was filtered. The solid filtrate was rinsed with several additional portions of Et₂O. The collected solid was dried in vacuo to provide 3.22 g (6.14 mMol, 96% yield) of a white powder.

HPLC (Method A) tᵣ=3.07 min; MS (ES⁺): m/z 452.3 (M+H).

### Example 6

### Preparation of I-2

¹H NMR (CD₃OD, 500 MHz) δ 8.18 (d, 1H), 7.60 (two d, 2H), 7.29 (dd, 2H), 6.31 (br s, 1H), 5.30 (d, 1H), 4.38 (br d, 1H), 4.10 (d, 1H), 3.88 (m, 2H), 3.73 (m, 1H), 3.66 (br s, 1H), 3.43 (dd, 1H), 3.32 (s, 1H), 2.33 (m, 1H), 2.22 (m, 1H), 2.13-1.93 (two m, 4H), 1.80 (m, 2H), 1.68 (d, 1H), 1.45-1.17 (m, 5H); HPLC (Method B) tᵣ=2.49 min; MS (ES⁺): m/z 452.3 (M+H).

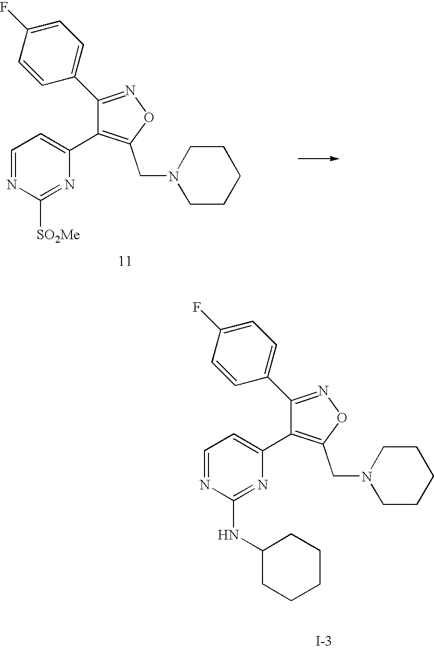

### Example 7

### Preparation of I-3

A stirred solution of 11 (22.65 g, 54.38 mMol) and cyclohexylamine (12.4 mL, 109 mMol) in DMSO (300 mL) was heated to 85° C. for 4 h. The reaction was cooled, poured into water and extracted with several portions of CH₂Cl₂. The combined organic layer was washed with brine, dried (MgSO₄), filtered and concentrated. The solid residue was recrystalized from EtOH to provide 18.75 g (43.05 mMol, 79%) of I-3.

¹H NMR (CDCl₃, 500 MHz) δ 8.18 (d, 1H, J=4.9 Hz), 7.52-7.48 (two d, 2H, J=8.8 Hz and 8.8 Hz), 7.11-7.07 (two d, 2H, J=8.7 and 8.7 Hz), 6.40 (br s, 1H), 5.00 (d, 1H, J=8.0 Hz), 3.94 (s, 2H), 3.68 (br s, 1H), 2.50 (m, 4H), 1.93 (m, 2H), 1.72 (m, 2H), 1.59 (m, 5H), 1.42 (m, 2H), 1.38-1.12 (two m, 5H).

bis-HCl salt: A solution of I-3 (1.32 g, 3.04 mMol) in MeOH—CH₂Cl₂ (1:1) was treated with an excess of HCl-Et₂O solution. After several minutes, the solution was concentrated and the volatile components were removed in vacuo to provide the corresponding bis-HCl salt (1.50 g, 2.95 mMol, 97% yield) as a white solid.

HPLC (Method A) tᵣ=3.25 min; MS (ES⁺): m/z 436.2 (M+H).

### Example 8

### Preparation of I-4 (TFA Salt)

¹H NMR (CD₃OD, 500 MHz) δ 8.28 (d, 1H), 7.59 (m, 2H), 7.28 (two d, 2H), 6.49 (br s, 1H), 5.05 (s, 2H), 3.70 (m, 1H) 3.54 (m, 4H), 2.25 (m, 4H), 1.97 (m, 2H), 1.82 (m, 2H), 1.78 (d, 1H), 1.40-1.20 (m, 5H); HPLC (Method B) tᵣ=2.55 min; MS (ES⁺): m/z 422.3 (M+H).

### Example 9

### Preparation of I-5

¹H NMR (CDCl₃, 500 MHz) δ 8.12 (d, 1H), 7.51 (m, 2H), 7.42 m, 3H), 6.35 (d, 1H), 5.08(d, 1H), 4.00 (s, 2H), 3.70 (br s, 1H), 2.51 (br s, 4H), 1.95 (m, 2H), 1.72 (m, 2H), 1.60 (m, 5H), 1.41 (m, 2H), 1.36-1.08 (two m, 5H); HPLC (Method B) tᵣ=2.53 min; MS (ES⁺): m/z 418.1 (M+H).

### Example 10

### Preparation of I-6 (TFA Salt)

¹H NMR (CD₃OD, 500 MHz) δ 8.20 (d, 1H), 7.55 (two d, 2H), 7.15 (dd, 2H), 6.20 (br s, 1H), 4.99 (m, 1H), 4.84 (s, 2H) 4.67 (s, 1H), 3.85-3.65 (m, 3H), 3.43 (m, 1H), 2.37 (s, 1H), 2.10 (m, 1H), 1.94 (br s, 2H), 1.86 (d, 2H), 1.63 (d, 1H), 1.42-1.12 (m, 5H); HPLC (Method B) tᵣ=2.50 min; MS (ES⁺): m/z 438.3 (M+H).

### Example 11

### Preparation of I-7 (TFA Salt)

¹H NMR (CD₃OD, 500 MHz) δ 8.22 (d, 1H), 7.62 (two d, 2H), 7.28 (dd, 2H), 6.17 (br s, 1H), 4.77 (d, 1H), 4.69 (d, 1H), 4.38 (br s, 1H), 3.78 (m, 2H), 3.43 (m, 2H), 3.18 (dd, 1H), 2.20 (dd, 1H), 2.06-1.93 (m, 2H), 1.83 (m, 4H), 1.70 (d, 1H), 1.45-1.20 (m, 5H); HPLC (Method B) tᵣ=2.63 min; MS (ES⁺): m/z 452.3 (M+H).

### Example 12

### Preparation of I-8

¹H NMR (CDCl₃, 500 MHz) δ 8.13 (d, 1H), 7.49 (m, 2H), 7.40 (m, 3H), 6.31 (d, 1H), 5.08 (d, 1H), 4.01 (s, 2H), 3.70 (br s, 2H), 2.38 (m, 2H), 2.37 (m 2H), 2.05-1.85 (m, 4H), 1.80-1.52 (two m, 5H), 1.42-1.11 (rwo m, 5H); HPLC (Method B) tᵣ=2.33 min; MS (ES⁺): m/z 434.1 (M+H).

### Example 13

### Preparation of I-9

¹H NMR (CDCl₃, 500 MHz) δ 8.08, (d, 1H), 7.55-7.38 (complex m, 5H), 6.68 (br s, 1H), 6.07 (br s, 1H), 4.39 (d, 1H), 3.93-3.42 (three m, 4H), 3.23 (br s, 1H), 2.82 (br s, 1H), 2.61 (dd, 1H), 2.07-1.68 (complex m, 8H), 1.60 (m, 1H), 1.38-1.15 (two m, 5H); HPLC (Method B) tᵣ=2.55 min; MS (ES⁺): m/z 434.1 (M+H).

### Example 14

### Preparation of I-10

¹H NMR (CDCl₃, 500 MHz) δ 8.21 (d, 1H), 7.52 (two d, 2H), 7.12 (dd, 2H), 6.50 (br s, 1H), 5.25 (br s, 1H), 3.98 (s, 2H), 3.80-3.43 (three m, 3H), 2.92 (d, 1H), 2.72 (br s, 1H), 2.38-2.10 (m, 2H), 2.10-1.80 (two m, 3H), 1.80-1.55 (two m, 6H), 1.40-1.05 (m, 6H); HPLC (Method B) tᵣ=2.51 min; MS (ES⁺): m/z 466.5 (M+H).

### Example 15

### Preparation of I-11

bicyclo-[2.2.1]hept-2-yl-[4-(3-phenyl-5-piperidin-1-ylmethyl-isoxazol-4-yl)-pyrimidin-2-yl]-amine: ¹H NMR (500 MHz, CDCl₃) δ 8.05 (1 H, d), 7.42 (2 H, d), 7.33 (3 H, m), 6.28 (1 H, d), 5.10 (1 H, br s), 3.95 (2 H, s), 3.55 (1 H, br s), 2.40 (4 H, br s), 2.2 (1 H, br s), 2.15 (1 H, br s)1.70 (1 H, m), 1.50 (5 H, m), 1.40 (2 H, br s), 1.35 (4 H, m), 1.05 (2 H, m); HPLC (Method A): 3.18 min; MS (ES⁺): m/z 430.27 (M+H).

### Example 16

### Preparation of I-12 (TFA Salt)

¹H NMR (CDCl₃, 500 MHz) δ 9.91 (m, 1H), 9.32-8.25 (br s, 2H), 8.08 (m, 1H), 7.45 (m, 2H), 7.22 (m, 2H), 6.31 (d, 1H), 5.08-4.80 (m, 2H), 3.82 (m 4H), 2.98 (m, 2H), 2.13-1.89 (complex m, 9H), 1.56 (dd, 2H), 1.42 (m, 3H); HPLC (Method C) tᵣ=5.59 min; MS (ES⁺): m/z 452.3 (M+H).

### Example 17

### Preparation of N—CBz-(1R,2R,4S)-bicyclo[2.2.1]hept-2-ylamine and N—CBz-(1S,2S,4R)-bicyclo[2.2.1]hept-2-ylamine

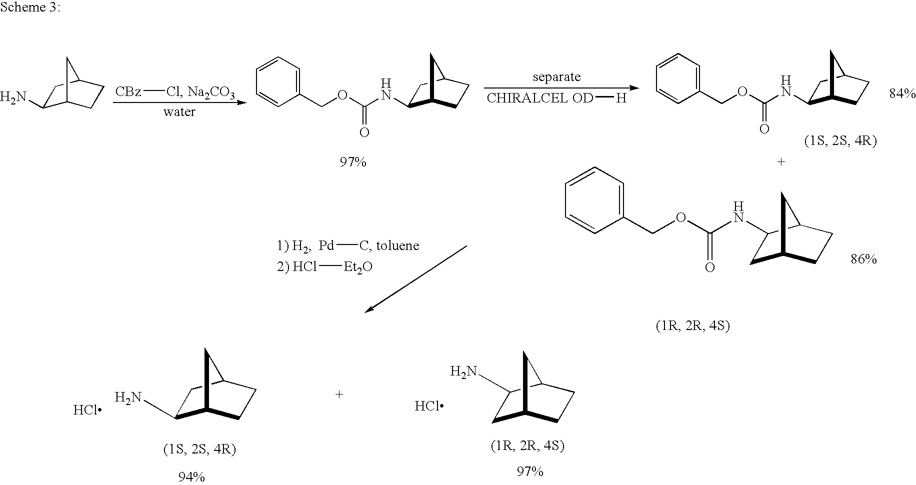

As depicted in Scheme 3, N—CBz-(1R,2R,4S)-bicyclo[2.2.1]hept-2-ylamine and N—CBz-(1S,2S,4R)-bicyclo[2.2.1]hept-2-ylamine (and the HCl salt as described in Example 18) for use in Examples 19, 20 and 21 are prepared as follows: To a solution of racemic 2-exo-aminonorbomane (22.0 g, 198 mMol) and Na₂CO₃ (22.0 g, 207 mMol) in water (220 mL) at 0° C., was added slowly via dropping funnel benzyl chloroformate (35.5 g, 198 mMol). After 20 min, additional water (100 mL) was added to the reaction and addition was continued for an additional 20 min. Upon complete addition, Et₂O (100 mL) was added and the reaction was maintained at 0° C. for 1 h. Then, the reaction was warmed to ambient temperature and poored into Et₂O (200 mL). The layers were partitioned and the aqueous layer was extracted with additional Et₂O (300 mL). The combined organic phase was washed with 1 M HCl, 1M NaOH, and brine, dried (MgSO₄), filtered throuh silica gel and concentrated to provide a white solid (47.0 g, 192 mMol, 97%).

This material (43.8 g) was resolved using preparative HPLC (Column: CHIRALCEL® OD-H®, 2.1×25 cm, eluent: CO₂/IPA:90/10, 30° C.). N—CBz-(1R,2R,4S)-bicyclo[2.2.1]hept-2-ylamine (18.94 g, 86.4% recovery, >99% ee) HPLC: tᵣ=3.51 min. N—CBz-(1S,2S,4R)-bicyclo[2.2.1]hept-2-ylamine (19.42 g, 84.1% recovery, 99% ee) HPLC: tᵣ₌3.90 min.

### Example 18

### Preparation of (1S,2S,4R)-bicyclo[2.2.1]hept-2-ylamine HCl Salt

A degassed solution of N—CBz-(1R,2R,4S)-bicyclo[2.2.1]hept-2-ylamine (19.42 g, 79.16 mMol) and Pd (5% on carbon, 1.9 g) in toluene at 0° C. was placed under H₂ atmosphere. The ice-bath was removed and the reaction was stirred for 19 h. Then, MeOH (100 mL) was added and the mixture was stirred for 15 min. The reaction was filtered through Celite and the cake was rinsed with MeOH (100 mL). The resulting filtrate was treated with HCl (2.0 M, 45 mL) and stirred for 10 min. Concentration of the liquid provided a white solid (11.0 g, 74.6 mMol, 94.2%). ¹H NMR (CD₃OD, 500 MHz) δ 3.13 (dd, 1H), 2.39 (m, 1H), 2.34 (d, 1H), 1.81 (ddd, 1H), 1.68-1.52 (complex m, 3H), 1.42 (dd, 1H), 1.34 (d, 1H), 1.23 (m, 2H).

### Example 19

### Preparation of I-13

A stirred solution of 12 (16.0 g, 37.1 mMol) and (1R,2R,4S)-bicyclo[2.2.1]hept-2-ylamine (The enantiopurity and absolute stereochemistry of the final products were asigned based on HPLC comparison with material prepared with (1R,2R,4S)-bicyclo[2.2.1]hept-2-ylamine or (1S,2S,4R)-bicyclo[2.2.1]hept-2-ylamine obtained by synthesis following literature methods. See: Eda, M; Takemoto, T.; Ono, S.-I.; Okada, T., Kosaka, K.; Gohda, M.; Matzno, S.; Nakamura, N.; Fukaya, C. J. Med. Chem.1994, 37, 1983-1990 and references therein) (6.75 g, 44.5 mMol) and Na₂CO₃ (4.72 g, 44.5 mMol) in DMSO (20 mL) was heated to 70° C. for 20 h. The reaction was allowed to cool to ambient temperature. The reaction was diluted with CH₂Cl₂, poured into water, partitioned extracted with two additional portions of CH₂Cl₂. The combined organic phase was washed with brine, dried (MgSO₄), filtered and concentrated. Flash chromatography (SiO₂, EtOAc eluent) provided 16.0 g of viscous oil. ¹H NMR of free base, same as I-18.

### Example 20

### Preparation of Bis HCl Salt

A solution of I-13 (16.0 g) in MeOH—CH₂Cl₂ (1:4) was treated with HCl (2.5-3 eq, 2.0 M in Et₂O). After a few min a precipitate started to form. After 30 minutes, additional Et₂O was added and the solution was filtered. The solid filtrate was rinsed with several additional portions of _{Et2O}. The collected solid was dried in vacuo to provide 15.69 g (29.25 mMol, 79% yield) of a white powder. This material could be further purified by suspending it in warm MeOH for 15 min, and precipitating the salt with addition of MTBE.

HPLC (Method A) tᵣ=2.79 min; HPLC (method C) tᵣ=4.60 min; HPLC (chiral Method D) tᵣ=14.74 min, (>99% ee); MS (ES⁺): m/z 464.2 (M+H).

### Example 21

### Preparation of I-14

was prepared as described for I-13 starting with (1S,2S,4R)-bicyclo[2.2.1]hept-2-ylamine. ¹H NMR of free base, same as I-18. HPLC (Method A) tᵣ=2.79 min; HPLC (method C) tᵣ=4.60 min; HPLC (chiral Method D) tᵣ=16.59 min, (98.2% ee); MS (ES⁺): m/z 464.3 (M+H).

### Example 22

### Preparation of I-15

¹H NMR (CDCl₃, 500 MHz) δ 8.18 (d, 1H), 7.50 (m, 2H), 7.11 (dd, 2H), 6.40 (br s, 1H), 5.10 (d, 1H), 4.12 (s, 2H), 3.77 (br s, 2.76 (m, 4H), 1.94 (br s, 2H), 1.72-1.53 (three m, 10H), 1.38-1.10 (two m, 5H); HPLC (Method B) tᵣ=2.90 min; MS (ES⁺): m/z 450.5 (M+H).

### Example 23

### Preparation of I-16

¹H NMR (CDCl₃, 500 MHz) δ 8.16 (d, 1H), 7.50 (m, 2H), 7.17 (m, 2H), 6.49 (br s, 1H), 4.96 (s, 2H), 3.53 (br s, 1H), 3.28 (s, 1H), 2.95 (d, 2H), 2.18 (m, 3H), 1.90-1.65 (complex m, 8H), 1.61 (m, 1H); HPLC (Method B) tᵣ=2.51 min; MS (ES⁺): m/z 479.5 (M+H).

### Example 24

### Preparation of I-17 (4-Methyl-cyclohexyl)-[4-(3-phenyl-5-piperidin-1-ylmethyl-isoxazol-4-yl)-pyrimidin-2-yl]-amine:

¹H NMR (500 MHz, CDCl₃) δ 8.05 (1 H, m), 7.4 (2 H, m), 7.30 (3 H, m), 6.25 (1 H, d), 5.22 (0.5 H, br s), 4.95 (0.5 H, br s), 3.95 (3 H, d), 3.55 (1 H, br s), 2.40 (4 H, br s), 1.95 (2 H, s), 1.90 (1 H, d), 1.65 (2 H, d), 1.50 (6 H, m), 1.35 (3 H, m), 1.10 (1 H, m), 0.90 (1 H, m); HPLC (Method A): 3.26 min; MS (ES⁺): m/z 432.33 (M+H).

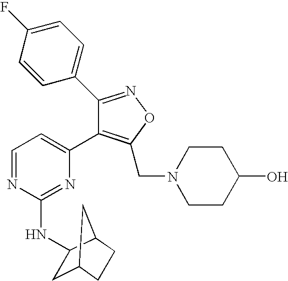

### Example 25

### Preparation of I-18

A stirred solution of 12 (2.00 g, 4.62 mMol) and 2-exo-nobornylamine (1.10 mL, 9.24 mMol) in DMSO (20 mL) was heated to 75° C. for 4 h. The reaction was allowed to cool to ambient temperature. The reaction was diluted with CH₂Cl₂, poured into water, partitioned extracted with two additional portions of CH₂Cl₂. The combined organic phase was washed with brine, dried (MgSO₄), filtered and concentrated. Flash chromatography (SiO₂, EtOAc eluent) provided 2.03 g (4.38 mMol, 95%) of a colorless foam.

¹H NMR (CDCl₃, 500 MHz) δ 8.17 (m, 1H), 7.52-7.48 (two d, 2H, J=8.8 Hz and 8.8 Hz), 7.12-7.07 (two d, 2H, J=8.7 and 8.6 Hz), 6.37 (d, 1H, J=5.1 Hz), 5.02 (d, 1H, J=5.7 Hz), 4.02 (s, 2H), 3.74-3.62 (m, 2H), 2.82 (m, 2H), 2.37-2.26 (m, 3H), 2.20 (br s, 1H), 1.90 (m,, 2H), 1.75 (br s, 1H), 1.61 (m, 2H), 1.49 (m, 2H), 1.41 (d, 1H, J=10.2 Hz), 1.36 (d, 1H, J=4.5 Hz), 1.36 1.25-1.11 (m, 4H).

Bis HCl salt: A solution of I-18 (2.03 g, 4.38 mMol) in MeOH—CH₂Cl₂ (1:2) was treated with HCl (17.5 mL, 1 M in Et₂O). After a few min a precipitate started to form. After 30 minutes, additional Et₂O was added and the solution was filtered. The solid filtrate was rinsed with several additional portions of Et₂O. The collected solid was dried in vacuo to provide 1.93 g (3.60 mMol, 82% yield) of a white powder.

HPLC (Method A) tᵣ=2.79 min; HPLC (method C) tᵣ=4.59 min; (chiral Method D) tᵣ=14.87 and 16.87 min; MS (ES⁺): m/z 464.3 (M+H).

### Example 26

### Preparation of I-19

¹H NMR (CDC]₃, 500 MHz) δ 8.27 (d, 1H), 7.49 (two d, 2H), 7.07 (dd, 2H), 6.40 (d, 1H), 5.07 (d, 1H), 4.96 (s, 2H), 3.65 (m, 1H), 3.38 (s, 2H), 2.48 (br s, 4H), 2.28 (s, 1H), 2.18 (br s, 1H), 1.77 (m, 1H), 1.57 (m, 4H), 1.53-1.37 (two m, 4H), 1.17-1.06 (two m, 4H); HPLC (Method B) tᵣ=2.70 min; MS (ES⁺): m/z 448.3 (M+H).

### Example 27

### Preparation of I-20 (Mixture of Diastereomers)

¹H NMR (CDCl₃, 500 MHz) δ 8.18 (d, 1H), 7.48 (m, 2H), 7.08 (dd, 2H), 6.35 (d, 1H), 5.28 and 4.98 (two d, 1H), 3.95 (d, 2H), 3.70 (m, 1H), 2.81 (m, 2H), 2.33 (dd, 2H), 1.96 (br s, 1H), 1.86 (m, 2H), 1.71 (d, 2H), 1.65-1.45 (complex m, 5H), 1.34 m, 1H), 1.17 (m, 2H), 1.00 (m, 1H), 0.91 (two d, 3H); HPLC (Method B) tᵣ=2.63 min; MS (ES⁺): m/z 466.3 (M+H).

### Example 28

### Rat MCAO Efficacy Studies

General Procedures

Rats were anesthetized with isoflurane and were prepared for sterile surgery. The MCA was occluded using the intraluminal technique to induce ischemia (Schmid-Elsaesser, etc, Stroke, 1998; 29:2162-2170). The occluder was removed at 2 hour post ischemia and rats were dosed with compound or vehicle using the Med-e-cell pumps provided by Vertex. Compounds were dosed either by I.P. injection or by I.V. injection, and were administered in the range of 1-100 mg/kg in 2, 3 or 4 dosage administrations. The i.v. bolus and constant infusion was administered through the external jugular vein (cannulated before MCAo). The total duration of the experiment was 24, 48 or 72 hours. At the end of the experiment the rat brains were removed, and chilled on ice in 1×PBS for 10 mins. Two mm thick coronal sections (7 sections per brain) were be stained by 2% TTC in 1×PBS and post fixed overnight by 10% neutral buffered formalin.

At 2-hr post ischemia, before the removing the occluder, a decision to include or exclude the animal in the study was made based on the neurological deficit criteria. A scale of 0 to 3 was used for each of the following behavioral responses: 1) rotation, 2) tactile whisker response and 3) forearm torsion upon tail suspension, calculate a response score (0-9) for each animal. A minimum score for inclusion required 5 or more. Additionally, any animals that die prematurely were excluded from the study. Additional animals were included in the study to ensure that the required final ‘N” in each group is obtained.

Physiological Variables Measured:

Body temperature was monitored throughout the surgery and maintained near normal values (36.8-37.5° C.). Body temperature was documented at the time of MCAO, two hours into ischemia, at the beginning of treatment (2, 4 or 6 hr post ischemia), 24, 48 and 72 hr post ischemia (end of experiment).

Body weight was documented at the 0, 24, 48 and 72 hr post ischemia.

Behavioral assessment was performed prior to ischemia, and at 2, 4, 6, 24, 48 and 72 hr post ischemia.

Compounds of the invention were administered in dosages in the range of 1-100 mg/kg in 3 or 4 dosage administrations 2, 4 or 6 hours after ischemia challenge. In general, compounds administered under these conditions exhibit % protection in the range of 10-70% protection.

In certain preferred embodiments compounds are administered 2 hours after ischemia challenge (TMCAO (transient MCAO) or PMCAO (permanent MCAO) model) in administration dosages ranging from 2-100 mg/kg (using 3 or 4 dosage administrations) and exhibit % protection in the range of about 35 to about 70.

In still other preferred embodiments, compounds are administered using the TMCAO model 4 or 6 hours after ischemia challenge in administration dosages ranging from 2-15 mg/kg (using 3 or 4 dosage administrations) and exhibit % protection in the range of about 30 to about 55.

In certain other preferred embodiments, compounds are administered using the TMCAO model 4 hours after ischemia challenge in administration dosages ranging from 2-100 mg/kg (using 3 or 4 dosage administrations) and exhibit % protection in the range of about 40 to about 55.

In still other preferred embodiments, compounds of the invention are dosed in a continuous infusion mode. In yet other preferred embodiments, compounds are dosed in a continuous infusion mode in the range of about 0.125 to about 5 mg/kg/hr.

### Example 29

### In Vitro Ischemia (OGD) Assay

Determination of the Percent Neuroprotection

As used herein, the term “Percent Protection” represents the percentage of neuronal cells protected against ischemic injury (OGD) and is calculated as:

% protection=(test−OGD)/(normal−OGD)*100

This protocol describes the procedure used to induce experimental ischemia by anoxia-re-oxygenation in cultured hippocampal neuronal cells. The neuroprotective effect of test compounds is evaluated against ischemic-induced neuronal cell injury and cell death

The following steps were performed prior to the day of the assay:

The LoG-Neurobasal [LoG-Neurobasal contains NoG-Neurobasal medium (Invitrogen Corp, customized order) plus 0.5 mM glucose, 0.5 mM L-glutamine and 0.25× Penicillin/Streptomycin] was pre-equilibrated in the hypoxic chamber overnight.

The LoG-Neurobasal was pre-equilibrated in the normal incubator (5% CO₂) overnight.

In the normal incubator (5% CO₂), Neurobasal/B27AO [Neurobasal/B27AO contains Neurobasal medium (Invitrogen Corp Cat #21103-049) with 2× B27 minus AO supplement (Invitrogen Corp Cat #10889-038), 0.5 mM L-glutamine, and 0.25× Penicillin/Streptomycin] was pre-equilibrated overnight.

The following steps were performed the day of the assay:

LoG-Neurobasal medium was removed from the hypoxic chamber, and the medium was lightly bubbled with 100% N₂ for 30 minutes to deoxygenate completely.

The Neurobasal/B27m culture medium [Neurobasal/B27m contains Neurobasal medium with 2× B27 supplement (Invitrogen Corp Cat #17504-044) and 0.5 mM L-glutamine] was aspirated from the cells in each 12-well plate using the vacuum pump with a sterile glass pastuer pipette attached.

The plate was washed once with 2 ml of glucose free-BSS₀ (pH 7.4), prepared from the following: 143.6 mM NaCl, 5.4 mM KCl, 1.8 mM CaCl₂, 0.8 mM MgSO₄, 1 mM NaH₂PO₄, 26.2 mM NaHCO₃, 10 mg/l phenol red, and 0.25×P/S.

The neurons (10-11 days from initial culture) were replenished with deoxygenated LoG-Neurobasal (1 ml per well for each well of a 12-well plate). These neuronal cells were prepared according to Park L C, Calingasan N Y, Uchida K, Zhang H, Gibson G E. (2000) “Metabolic impairment elicits brain cell type-selective changes in oxidative stress and cell death in culture.” J Neurochem 74(1):114-124.

The test compounds were added directly to each well (3 concentrations of the compound plus positive control, each in triplicate). The compounds were dissolved in 100% DMSO, where the concentration of DMSO never exceeded 0.5% then the plates were placed in the Hypoxic Chamber for 5 hours with the plate lids ajar.

For normoxia controls, pre-equilibrated normoxic LoG-Neurobasal medium was added to each well and the plate replaced in the normal culture incubator for 4 hours.

After 4 hours of hypoxia, the existing media was carefully aspirated off and 2 mL of new oxygenated (pre-equilibrated) Neurobasal/B27AO was added to each well. Re-oxygenated medium was achieved by placing medium overnight in the culture incubator (5% CO₂/95% O₂) prior to use.

The same test compounds with same the concentrations were added back into the corresponding wells and the plates placed in the cell culture incubator (5% CO₂/95% O₂) and re-oxygenated for 20-24 hours. After re-oxygenation for 20-24 hours, the number of live neurons are counted using the cell tracker green fluorescence method described below.

The existing culture medium was aspirated from each well of the 12 well plates and the neurons were washed once with 2 ml of HBSS (pH 7.4, Invitrogen Corp, Cat #14170-112) pre-warmed to 30-37° C.

To each well of the plate was added 1 ml of 2.5 μM Cell Tracker Green ((Molecular Probes Cat #2925) and 5 μM Hoechst 33342 fluorescent dyes dissolved in HBSS. The plates were then placed in the dark at room temperature for 15 minutes then the neurons were washed once with 2 ml of HBSS. 1 ml of EBSS was added to each well, and the numbers of live and dead fluorescent cells were counted using Cellomics® automated imaging system.

In preferred embodiments, the following compounds were found to have a percent protection value of ≧50%: I-1, I-3, I-13 and I-18.

### Example 30

### In Vitro CNS Inflammation Assay

This protocol describes the procedure used to induce experimental inflammation by lipopolysacchride (LPS) in cultured CNS mixed glial cells. The anti-inflammatory effect of test compounds was evaluated against LPS-induced tumor necrosis factor-α (TNF-α) production in mixed glial cells.

The following steps were performed the day of the assay:

The mixed glial cells (7 days from initial culture) were replenished with mixed glial culture media consist of 50:50:10:1 combination of DMEM (high glucose: Invitrogen Corp), F-12 (Invitrogen Corp), 100% fetal bovine serum and 100×N-2 supplement (Invitrogen Corp). These mixed glial cells were prepared according to Park L C, Calingasan N Y, Uchida K, Zhang H, Gibson G E. (2000) “Metabolic impairment elicits brain cell type-selective changes in oxidative stress and cell death in culture.” J Neurochem 74(1):114-124 with some modification. Briefly, rat fetal forebrains (1-2 day old) were isolated, triturated and plated on 96-well plates (20,000 cells per well) in mixed glial media in 5% CO₂ incubator at 37° C. The cells were replenished with new media at 4^{th} day from initial culture.

The test compounds were added directly to each well (5 concentrations of the compound plus positive control, each in quadriplicate). The compounds were dissolved in 100% DMSO, where the concentration of DMSO never exceeded 0.5%. At 30 min after dosing, lipopolysacchrides (LPS) at 50 ng/ml were added directly to each well, and then the plates were placed in 5% CO₂ incubator at 37° C. for 6 hours. After 6 hours of LPS treatment, the existing media was carefully collected to detect the amount of TNF-α in the media.

The quantitative determination of TNF-αpresent in cell culture medium produced by mixed glial cells was performed using a solid phase sandwich enzyme linked immno-sorbent assay (ELISA) method based on the protocol and reagents provided by Biosource rat TNF-α ELISA kit (Biosource International).

In preferred embodiments, the following compounds were found to have IC₅₀s of 1 μM or less: I-1, I-13, I-14 and I-18. In other preferred embodiments, the following compounds were found to have IC₅₀s of 300 nM or less: I-13, I-14 and I-18.

1. A compound of formula I: 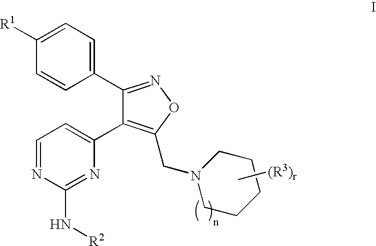
or a pharmaceutically acceptable salt thereof, wherein:
R¹ is hydrogen or halogen;
R² is substituted or unsubstituted cycloalkyl;
each occurrence of R³ is independently halogen, alkyl, —(CH₂)ₘOR⁴, —(CH₂)ₘSR⁴, —(CH₂)ₘN(R⁴)₂, —(CH₂)ₘNR⁴C(O)R⁴, —(CH₂)ₘNR⁴C(O)N(R⁴)₂, —(CH₂)ₘNR⁴CO₂R⁴, —(CH₂)ₘCO₂R⁴, —(CH₂)ₘC(O)R⁴, —(CH₂)ₘC(O)N(R⁴)₂, —(CH₂)ₘOC(O)N(R⁴)₂, —(CH₂)ₘS(O)₂R⁴, —(CH₂)ₘSO₂N(R⁴)₂, —(CH₂)ₘS(O)R⁴, —(CH₂)ₘNR⁴SO₂N(R⁴)₂, —(CH₂)ₘNR⁴SO₂R⁴, —(CH₂)ₘC(═S)N(R⁴)₂, wherein m is 0, 1 or 2 and R⁴ is hydrogen or alkyl;
r is 0, 1 or 2; and
n is 0, 1 or2.

2. The compound of claim 1, wherein R¹ is hydrogen or fluorine; R² is substituted or unsubstituted cycloalkyl; r is 0 or 1; R³ is alkyl, or —(CH₂)ₘOR⁴, wherein m is 0, 1 or 2 and R⁴ is hydrogen or alkyl; and n is 0, 1 or 2.

3. The compound of claim 1, wherein R² is substituted or unsubstituted norbornyl and compounds have the formula II: 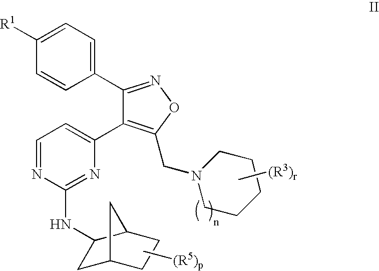
wherein R¹ is hydrogen or halogen;
each occurrence of R³ is independently halogen, alkyl, —(CH₂)ₘOR⁴, —(CH₂)ₘSR⁴, —(CH₂)ₘN(R⁴)₂, —(CH₂)ₘNR⁴C(O)R⁴, —(CH₂)ₘNR⁴C(O)N(R⁴)₂, —(CH₂)ₘNR⁴CO₂R⁴, —(CH₂)ₘCO₂R⁴, —(CH₂)ₘC(O)R⁴, —(CH₂)ₘC(O)N(R⁴)₂, —(CH₂)ₘOC(O)N(R⁴)₂, —(CH₂)ₘS(O)₂R⁴, —(CH₂)ₘSO₂N(R⁴)₂, —(CH₂)ₘS(O)R⁴, —(CH₂)ₘNR⁴SO₂N(R⁴)₂, —(CH₂)ₘNR⁴SO₂R⁴, —(CH₂)ₘC(═S)N(R⁴)₂, wherein m is 0, 1 or 2 and R⁴ is hydrogen or alkyl;
n is 0, 1 or 2;
r is 0, 1 or 2;
each occurrence of R⁵ is independently halogen, alkyl, —(CH₂)_{q}OR⁶, —(CH₂)_{q}SR⁶, —(CH₂)_{q}N(R⁶)₂, —(CH₂)_{q}NR⁶C(O)R⁶, —(CH₂)_{q}NR⁶C(O)N(R⁶ )₂, —(CH₂)_{q}NR⁶CO₂R⁶, —(CH₂)_{q}CO₂R⁶, —(CH₂)_{q}C(O)R⁶, —(CH₂)_{q}C(O)N(R⁶)₂, —(CH₂)_{q}OC(O)N(R⁶)₂, —(CH₂)_{q}S(O)₂R⁶, —(CH₂)_{q}SO₂N(R⁶)₂, —(CH₂)_{q}S(O)R⁶, —(CH₂)_{q}NR⁶SO₂N(R⁶)₂, —(CH₂)_{q}NR⁶SO₂R⁶, —(CH₂)_{q}C(═S)N(R⁶)₂, wherein q is 0, 1 or 2, and each occurrence of R⁶ is independently hydrogen or alkyl; and
p is 0, 1 or 2.

4. The compound of claim 1, wherein R² is substituted or unsubstituted cyclohexyl and compounds have the formula III: 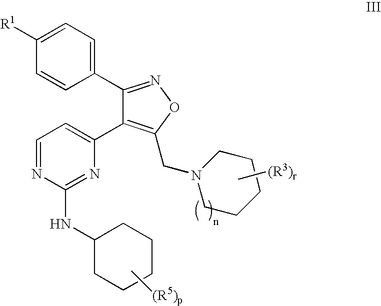
wherein R¹ is hydrogen or halogen;
each occurrence of R³ is independently halogen, alkyl, —(CH₂)ₘOR⁴, —(CH₂)ₘSR⁴, —(CH₂)ₘN(R⁴)₂, —(CH₂)ₘNR⁴C(O)R⁴, —(CH₂)ₘNR⁴C(O)N(R⁴)₂, —(CH₂)ₘNR⁴CO₂R⁴, —(CH₂)ₘCO₂R⁴, —(CH₂)ₘC(O)R⁴, —(CH₂)ₘC(O)N(R⁴)₂, —CH₂)ₘOC(O)N(R⁴)₂, —(CH₂)ₘS(O)₂R⁴, —(CH₂)ₘSO₂N(R⁴)₂, —(CH₂)ₘS(O)R⁴, —(CH₂)ₘNR⁴SO₂N(R⁴)₂, —(CH₂)ₘNR⁴SO₂R⁴, —(CH₂)ₘC(═S)N(R⁴)₂, wherein m is 0, 1 or 2 and R⁴ is hydrogen or alkyl;
n is 0, 1 or 2;
r is 0, 1 or 2;
each occurrence of R⁵ is independently hydrogen, halogen, alkyl, —(CH₂)_{q}OR⁶, —(CH₂)_{q}SR⁶, —(CH₂)_{q}N(R⁶)₂, —(CH₂)_{q}NR⁶C(O)R⁶, —(CH₂)_{q}NR⁶C(O)N(R⁶)₂, —(CH₂)_{q}NR⁶CO₂R⁶, —(CH₂)_{q}CO₂R⁶, —(CH₂)_{q}C(O)R⁶, —(CH₂)_{q}C(O)N(R⁶)₂, —(CH₂)_{q}OC(O)N(R⁶)₂, —(CH₂)_{q}S(O)₂R⁶, —(CH₂)_{q}SO₂N(R⁶)₂, —(CH₂)_{q}S(O)R⁶, —(CH₂)_{q}NR⁶SO₂N(R⁶)₂, —(CH₂)_{q}NR⁶SO₂R⁶, —(CH₂)_{q}C(═S)N(R⁶)₂, wherein q is 0, 1 or 2 and each occurrence of R⁶ is independently hydrogen or alkyl; and
p is 0, 1 or 2.

5. The compound of claim 1, wherein R¹ is F.

6. The compound of claim 1, wherein R¹ is H.

7. The compound of claim 1, wherein r is 0 or r is 1 and R³ is alkyl, OH, CH₂OH, or alkoxy.

8. The compound of claim 1, wherein n is 0.

9. The compound of claim 1, wherein n is 1.

10. The compound of claim 1, wherein n is 2.

11. The compound of claim 3, wherein p is 0.

12. The compound of claim 4, wherein p is 0.

13. The compound of claim 4, wherein p is 1.

14. The compound of claim 4, wherein p is 2.

15. The compound of claim 3 or 4, wherein p is 0 or 1 and R⁵ is OH, or alkyl.

16. The compound of claim 3, wherein R¹ is F or H; p is 0; n is 0 or 1; r is 0 or 1; and R³ is OH, CH₂OH, alkyl or alkoxy.

17. The compound of claim 4, wherein R¹ is F or H; p is 0, 1 or 2; each occurrence of R⁵ is independently alkyl, OH, CH₂OH or alkoxy; n is 0 or 1; r is 0 or 1; and R³ is OH, CH₂OH, alkyl or alkoxy.

18. The compound of claim 1, wherein the compound has one of the following structures:

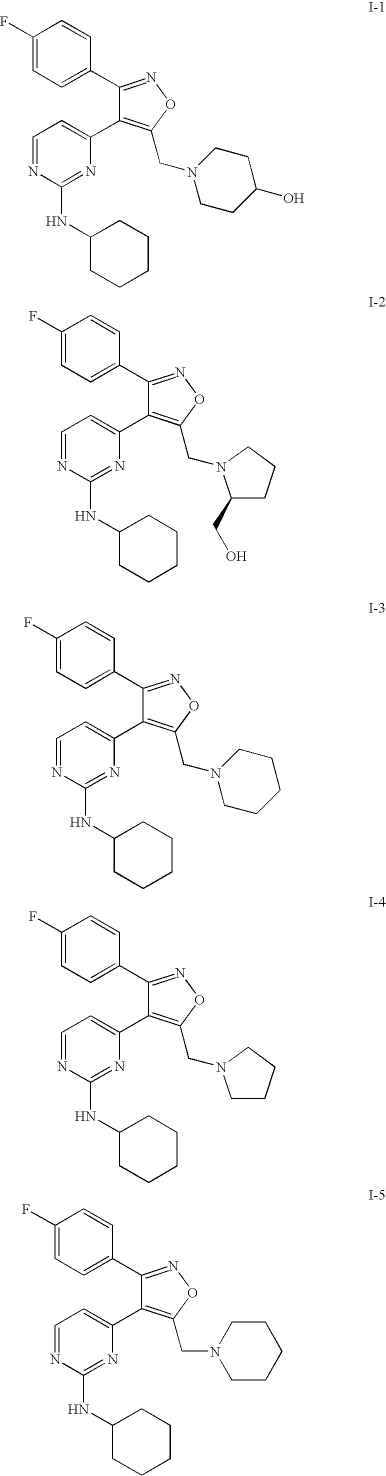

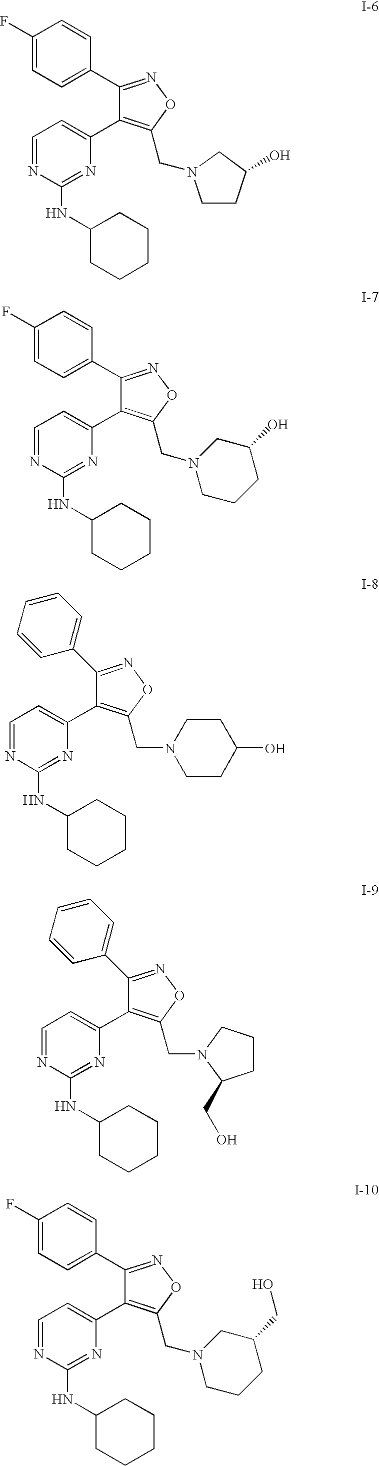

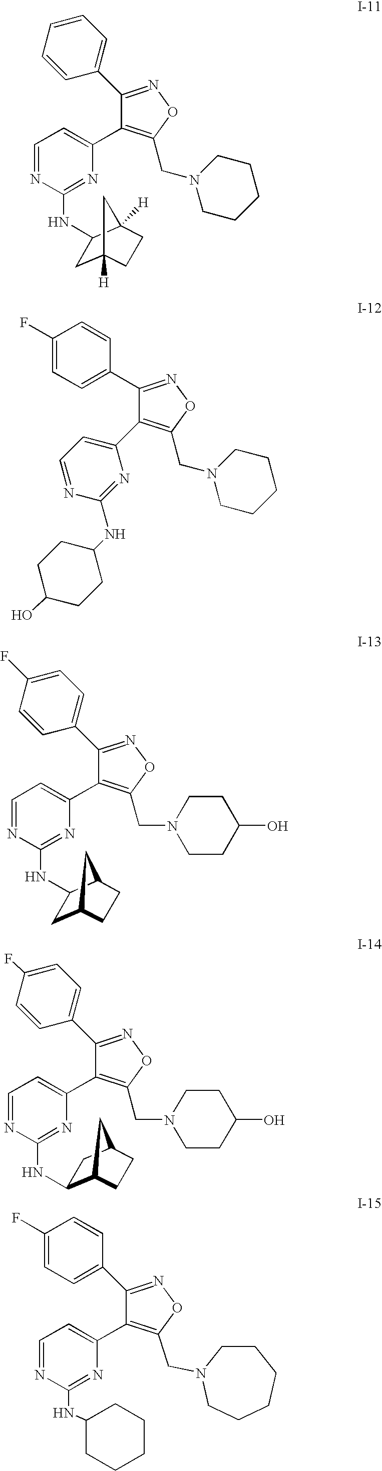

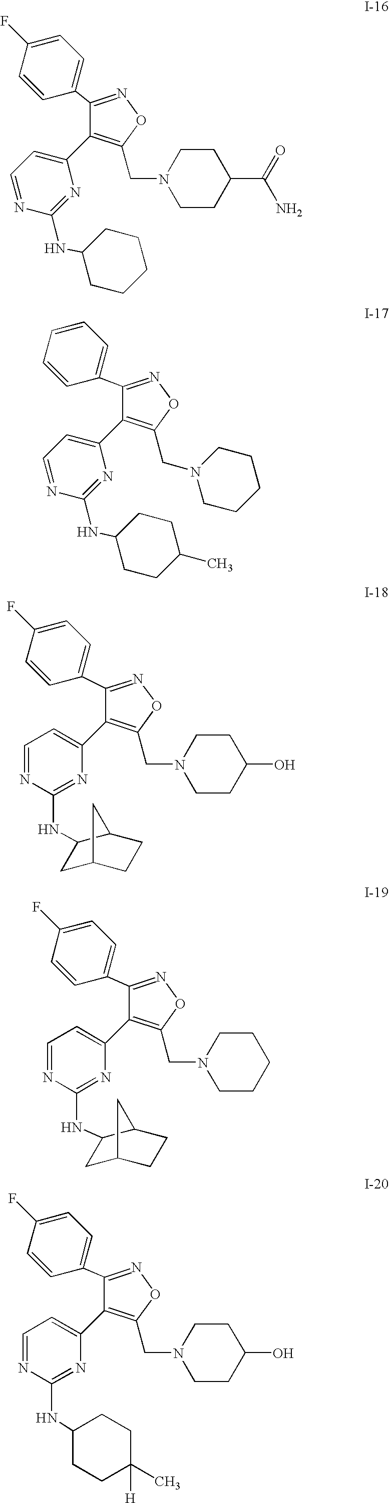

19. A pharmaceutical composition comprising a therapeutically effective amount of a compound of claim 1 and a pharmaceutically acceptable carrier or diluent.

20. The composition according to claim 19, further comprising an additional therapeutic agent selected from a treatment for stroke, a treatment for Alzheimer's Disease, a treatment for Parkinson's Disease, an agent for treating Multiple Sclerosis (MS), a treatment for asthma, an agent for treating schizophrenia, an anti-inflammatory agent, an immunomodulatory or immunosuppressive agent, a neurotrophic factor, an agent for treating cardiovascular disease, or an agent for treating an immunodeficiency disorder.

21. A method of treating a neurodegenerative, neurological, ischemic or inflammatory disorder comprising administering a therapeutically effective amount of a compound of claim 1.

22. The method according to claim 21, wherein the ischemic disorder is stroke.

23. The method according to claim 22, comprising the further step of:
administering to said patient an additional therapeutic agent selected from a treatment for stroke, a treatment for Alzheimer's Disease, a treatment for Parkinson's Disease, an agent for treating Multiple Sclerosis (MS), a treatment for asthma, an agent for treating schizophrenia, an anti-inflammatory agent, an immunomodulatory or immunosuppressive agent, a neurotrophic factor, an agent for treating cardiovascular disease, or an agent for treating an immunodeficiency disorder wherein:
said additional therapeutic agent is appropriate for the disease being treated; and
said additional therapeutic agent is administered together with said composition as a single dosage form or separately from said composition as part of a multiple dosage form.
